(19) United States
(12) Patent Application Publication
(10) Pub. No.: US 2012/0136164 A1
(43) Pub. Date: May 31, 2012

(54) NANOSTRUCTURED METALS

(75) Inventors: Jackie Y. Ying, Singapore (SG); Nandanan Erathodiyil, Singapore (SG); Hongwei Gu, Singapore (SG); Huilin Shao, Singapore (SG); Jiang Jiang, Singapore (SG)

(73) Assignee: Agency for Science, Technology and Research, Connexis (SG)

(21) Appl. No.: 13/260,968

(22) PCT Filed: Mar. 30, 2010
(86) PCT No.: PCT/SG2010/000124

§ 371 Date: Feb. 17, 2012

(30) Foreign Application Priority Data
Mar. 30, 2009 (SG) 200902179-1

(51) Int. Cl. C07D 307/33; C07C 303/36; C07C 29/145; B01J 35/02; B01J 23/89; B01J 37/04; C30B 7/14; C07C 67/30; B01J 23/42; B82Y 30/00; B82B 1/00; B82B 3/00; B82Y 99/00; B82Y 40/00

(52) U.S. Cl. 549/319; 560/179; 560/12; 568/814; 560/60; 502/300; 502/326; 502/339; 117/68; 977/762; 977/888; 977/840; 977/902

(57) ABSTRACT

The invention relates to a nanoparticulate material comprising long ultrathin metal nanowires, and to processes for making it. The nanoparticulate material may be used as a catalyst and, in the presence of a chiral modifier, can catalyse enantioselective reactions.

### TECHNICAL FIELD

The present invention relates to nanostructured metals and their use in catalysis.

### BACKGROUND OF THE INVENTION

The importance of optically pure compounds in the pharmaceutical, agricultural and fine chemicals industries has increased tremendously in recent years. Although homogeneous catalysts and ligands remain the usual choice, the development of efficient heterogeneous catalysts has attracted great interest in organic chemistry. Hydrogenation is of particular significance due to its potential to produce a variety of biologically and pharmaceutically important molecules, intermediates and specialty chemicals by molecular hydrogen under green reaction conditions. Even though there are several highly active and selective homogeneous catalysts known in the literature, only a few are used industrially due to limitations such as high cost, difficulties in catalyst recycling, product contamination, toxicity of metals and ligands. Heterogeneous catalysts would allow for ease of catalyst recovery and reuse, and applicability to continuous flow systems, which provide for cost-effectiveness and ease of scale-up and product isolation.

Enantioselective hydrogenation is one of the most important industrial asymmetric processes to produce chiral molecules with excellent selectivity (Scheme 1).

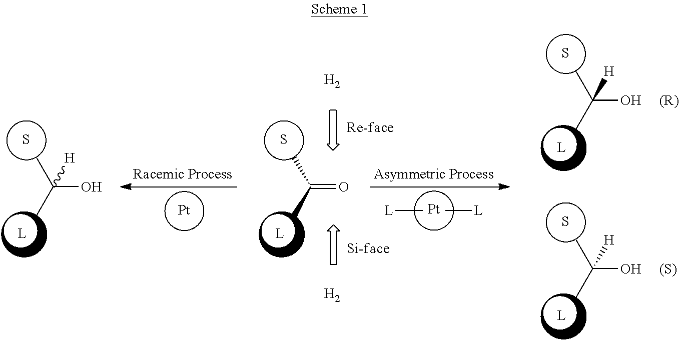

Catalyst modification is a strategy widely applied in heterogeneous catalytic hydrogenations. However, this strategy has been successful only in a limited number of reactions due to the high substrate specificity of such catalysts, i.e. only a particular combination of a metal, a modifier and a substrate type would give rise to good enantioselectivity. Metal nanostructures are of particular interest in this case because of their high activity under mild conditions associated with their large surface area, and because of their selectivity for catalytic transformations. Small variations in the metal, the modifier and the substrate type can lead to significant changes in enantiodiscrimination.

Catalytic hydrogenation of activated ketones is extremely important because of its effectiveness in producing chiral secondary alcohols. Nanostructures are of particular interest in the asymmetric hydrogenation of a-ketoesters. Platinum nanoparticle catalysts supported on silica, alumina and titania are mainly used in the hydrogenation of activated β-ketoesters. The activity and selectivity of the platinum catalyst are influenced by the support and chiral modifiers, e.g. cinchona alkaloids. Cinchona alkaloids have gained industrial importance in the enantioselective heterogeneous catalytic hydrogenations. Platinum catalysts modified with cinchona alkaloids for the hydrogenation of activated ketones have demonstrated ligand acceleration with a heterogeneous catalyst system, the Orito's catalytic system, giving reasonably good yield and selectivity (Scheme 2). [(a) von Arx, M.; Mallat, T.; Baiker, A. Top. Catal. 2002, 19, 75. (b) Vayner, G.; Houk, K. N.; Sun, Y.-K. J. Am. Chem. Soc. 2004, 126, 199]. However, the reproducibility and mechanism of this reaction have remained a challenge for this catalytic system.

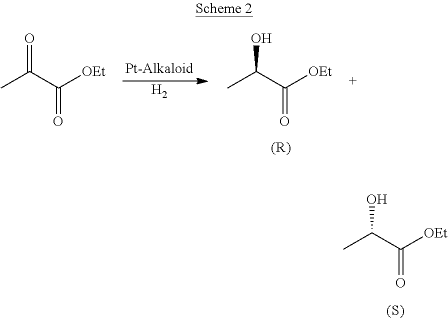

Nanowires and nanorods have received tremendous attention in recent years due to their applications in solar cells and other energy applications. Even though metal nanowires are known for many years, their application in catalysis, especially asymmetric catalysis, has not been explored.

### OBJECT OF THE INVENTION

It is the object of the present invention to substantially overcome or at least ameliorate one or more of the above disadvantages.

### SUMMARY OF THE INVENTION

In a first aspect of the invention there is provided a nanoparticulate material comprising (optionally consisting or consisting essential of) metal nanowires. The nanowires may be long ultrathin nanowires. The nanowires may have a diameter of less than about 2 nm. They may have a length of greater than about 40 nm, or greater than about 50 nm. Each nanowire may be a single crystal. The nanowires may be single crystal nanowires. The nanowires may be etched nanowires, optionally acid etched nanowires. The invention therefore provides, in an embodiment, a nanoparticulate material comprising (optionally consisting of, or consisting essential of) long ultrathin etched metal nanowires. It provides, in another embodiment, a nanoparticulate material comprising (optionally consisting of, or consisting essential of) long ultrathin single crystal metal nanowires.

The following options may be used in conjunction with the first aspect, either individually or in any suitable combination.

The nanowires may a length of about 40 to about 500 nm. They may have a length of 50 to about 500 nm. They may have a length of about 100 to about 500 nm. They may have a length greater than 500 nm. They may have a length of about 1 to about 10 microns. They may have a diameter of less than or equal to about 1.5 nm. They may have a diameter of less than or equal to about 1 nm. They may have a length of about 50 to about 500 nm and a diameter of less than about 2, optionally 1.5, nm.

The metal of the metal nanowires may be a Group 8 to Group 11 element, or may be a mixture of any two or more (e.g. 2, 3, 4 or 5) Group 8 to Group 11 elements. The metal may be for example platinum, palladium, rhodium, ruthenium or gold or a mixture of any two or more of these. In a particular embodiment the metal is platinum or is predominantly platinum.

The nanoparticulate material may be catalytic. It may be catalytic for a hydrogenation reaction.

The metal nanowires may have a chiral modifier associated therewith. The chiral modifier may be any suitable chiral compound for example an alkaloid (e.g. a Cinchona alkaloid), an optically active aminoalcohol, an optically active diamine, an optically active phosphine or an optically active aminophosphine or may be a mixture of any two or more of these. Examples of suitable chiral modifiers include 8R,9S-cinchonidine, 8R,9S-dihydrocinchonidine, 8R,9S-quinine, 8R,9S-dihydroquinine, 8S,9R-cinchonidine, 8S,9R-dihydrocinchonidine, 8S,9R-quinine and 8S,9R-dihydroquinine.

In an embodiment there is provided a catalytic nanoparticulate material comprising platinum nanowires having a diameter of less than about 2 nm and a length of about 50 to about 500 nm, optionally about 100 to about 500 nm. The nanowires may be straight nanowires. They may be nanorods.

In another embodiment there is provided a catalytic nanoparticulate material comprising platinum nanowires having a diameter of less than about 2 nm and a length of about 50 to about 500 nm, optionally about 100 to about 500 nm, and having a chiral modifier associated therewith (e.g. adsorbed thereon).

In another embodiment there is provided a nanoparticulate material for use in asymmetric hydrogenation reactions, said material comprising platinum nanowires having a diameter of less than about 2 nm and a length of about 50 to about 500 nm, optionally about 100 to about 500 nm, and having a chiral modifier associated therewith, said chiral modifier being an alkaloid.

In a second aspect of the invention there is provided a process for making a nanoparticulate material comprising:

a) preparing a mixture of a precursor and an amine, said precursor being capable of being converted to a metal or a mixture of metals; and
b) exposing the mixture to a metal carbonyl at elevated temperature; so as to produce the nanoparticulate material in the form of metal nanowires.
The process may produce the nanoparticulate material of the first aspect.

The following options may be used in conjunction with the second aspect, either individually or in any suitable combination.

The precursor may be a precursor to a metal selected from the Group 8 to Group 11 elements, or it may be a mixture of two or more such precursors. The precursor or, in the event that the more than one precursor is used, at least one of the precursors (or each independently) may be a metal complex. The complex may be for example an acetylacetone (acac) complex. In one embodiment, the complex is Pt(acac)₂.

The amine may be a C6 to C18 amine. It may be an alkenylamine. It may be for example oleylamine. The amine may function as a reducing agent. It may function as a surfactant. It may function as a reducing agent and as a surfactant. In the event that the amine is not capable of acting as a surfactant, a non-amine surfactant (e.g. a non-ionic surfactant) may be used in addition to the amine.

The process may be conducted under an inert atmosphere, e.g. a noble gas.

The metal carbonyl may be added in a trace amount (e.g. less than about 10% relative to the precursor on a molar basis with respect to the metals) or it may be added in a non-trace amount (e.g. greater than about 10%, optionally greater than about 100% relative to the precursor, on a molar basis with respect to the metals). The metal carbonyl may be for example iron pentacarbonyl.

The elevated temperature is between about 100 and about 300° C.

The process may additionally comprise the step of treating the nanowires with an etchant capable of removing the metal of the metal carbonyl. The etchant may be an acid. It may be a mineral acid. It may be for example hydrochloric acid. This option may be used when the metal carbonyl is used in greater than about 100%, on a molar basis with respect to the metals. This option may be capable of producing nanowires that have a diameter less than about 1.5 nm, optionally less than about 1 nm.

The mixture produced in step a) may also comprise a carboxylic acid salt. This option may be used when the metal carbonyl is used in less than about 10% on a molar basis with respect to the metals. The carboxylic acid may be a C6 to C18 carboxylic acid salt. It may be an alkenoic acid salt. It may be for example an oleate such as sodium oleate. The hydrocarbon group of the carboxylic acid salt may be the same as the hydrocarbon group of the amine or it may be different thereto. This option may be capable of producing straight nanowires.

The process may additionally comprise exposing the metal nanowires to a chiral modifier. The chiral modifier may be an alkaloid (e.g. a Cinchona alkaloid), an optically active aminoalcohol, an optically active amino acid, an optically active diamine, an optically active phosphine or an optically active aminophosphine or may be a mixture of any two or more of these. Suitable chiral modifiers include 8R,9S-cinchonidine, 8R,9S-dihydrocinchonidine, 8R,9S-quinine, 8R,9S-dihydroquinine, 8S,9R-cinchonidine, 8S,9R-dihydrocinchonidine, 8S,9R-quinine and 8S,9R-dihydroquinine.

The process may comprise:

a) preparing a mixture of a precursor and an amine, said precursor being capable of being converted to a metal or a mixture of metals; and
b) exposing the mixture to a metal carbonyl at elevated temperature; so as to produce the nanoparticulate material in the form of metal nanowires.
wherein:

if the metal carbonyl is used in less than about 10% on a molar basis with respect to the metals, the mixture produced in step a) also comprises a carboxylic acid salt and the nanowires produced by the process are straight, and

if the metal carbonyl is used in greater than about 100%, on a molar basis with respect to the metals, the process additionally comprises the step of treating the nanowires with an etchant capable of removing the metal of the metal carbonyl and the nanowires produced by the process have a diameter of less than about 1.5 nm, optionally less than about 1 nm.

In an embodiment there is provided a process for making a nanoparticulate material comprising:

a) preparing a mixture of a precursor, an amine and a carboxylate salt, said precursor being capable of being converted to a metal or a mixture of metals; and
b) exposing the mixture to a trace amount (e.g. less than 10% on a molar basis with respect to the metals of the precursor and the metal carbonyl) of metal carbonyl at elevated temperature;
so as to produce the nanoparticulate material in the form of metal nanowires.

In another embodiment there is provided a process for making a nanoparticulate material comprising:

a) preparing a mixture of a precursor and an amine, said precursor being capable of being converted to a metal or a mixture of metals;
b) exposing the mixture to a metal carbonyl at elevated temperature to form nanowires; and
c) treating the nanowires with an etchant capable of removing the metal of the metal carbonyl;
so as to produce the nanoparticulate material in the form of metal nanowires.

In another embodiment there is provided a process for making a nanoparticulate material comprising:

a) preparing a mixture of platinum complex and a C6 to C18 amine, said platinum complex being capable of being converted to a metal or a mixture of metals; and
b) exposing the mixture to iron pentacarbonyl at elevated temperature;
so as to produce the nanoparticulate material in the form of metal nanowires.

In another embodiment there is provided a process for making a nanoparticulate material comprising:

a) preparing a mixture of platinum complex and a C6 to C18 amine, said platinum complex being capable of being converted to a metal or a mixture of metals;
b) exposing the mixture to iron pentacarbonyl at elevated temperature; and
c) treating the nanowires with an acid capable of removing the iron;
so as to produce the nanoparticulate material in the form of metal nanowires.

In another embodiment there is provided a process for making a nanoparticulate material comprising:

a) preparing a mixture of platinum complex, a C6 to C18 carboxylate salt and a C6 to C18 amine, said platinum complex being capable of being converted to a metal or a mixture of metals; and
b) exposing the mixture to iron pentacarbonyl at elevated temperature;
so as to produce the nanoparticulate material in the form of metal nanowires.

The invention also provides a nanoparticulate material made by the process of the second aspect. Thus there is provided a nanoparticulate material made by:

a) preparing a mixture of a precursor and an amine, said precursor being capable of being converted to a metal or a mixture of metals; and
b) exposing the mixture to a metal carbonyl at elevated temperature;
so as to produce the nanoparticulate material in the form of metal nanowires.

In particular there is provided a nanoparticulate material made by:

a) preparing a mixture of a precursor, an amine and a carboxylate salt, said precursor being capable of being converted to a metal or a mixture of metals; and
b) exposing the mixture to a trace amount (e.g. less than 10% on a molar basis with respect to the metals of the precursor and the metal carbonyl) of metal carbonyl at elevated temperature;
so as to produce the nanoparticulate material in the form of metal nanowires.

There is also provided a nanoparticulate material made by:

a) preparing a mixture of a precursor and an amine, said precursor being capable of being converted to a metal or a mixture of metals;
b) exposing the mixture to a metal carbonyl at elevated temperature to form nanowires; and
c) treating the nanowires with an etchant capable of removing the metal of the metal carbonyl;
so as to produce the nanoparticulate material in the form of metal nanowires.

In a third aspect of the invention there is provided a method for conducting a catalytic reduction comprising exposing a substrate to a nanoparticulate material according the first aspect, or made by the process of the second aspect, in the presence of a hydrogen source. The nanoparticulate material may function as a catalyst. It may be a catalytic nanoparticulate material.

The following options may be used in conjunction with the third aspect, either individually or in any suitable combination.

The method may be conducted in an aqueous solvent.

The step of exposing may be conducted in the presence of a chiral modifier. The chiral modifier may be as described earlier.

The metal nanowires may be for example platinum nanowires, platinum/ruthenium nanowires or platinum/iron nanowires.

The hydrogen source may be hydrogen gas. The hydrogen gas may be at a pressure of less than about 750 kPa.

The hydrogen source may be ammonium formate. It may be alkaline isopropanol.

The nanowires of the nanoparticulate substance may have a chiral modifier associated therewith. The method may be enantioselective. It may be enantioselective across a wide range of substrates. The method may produce an optically active product. The chiral modifier may be as discussed above. The optically active product may have an enantiomeric excess of at least about 50%, or of at least about 60%. The chiral modifier may be a naturally occurring product such as an alkaloid, e.g. a cinchona alkaloid, or a protonated form thereof. In particular, in the event that the reaction is conducted in an aqueous solvent and the nanowires of the nanoparticulate substance have a chiral modifier associated therewith, the chiral modifier may be a protonated form of a basic chiral compound, for example a protonated alkaloid. Thus an acid may be added to the reaction mixture in order to protonate the chiral modifier. The acid may be added in at least about one molar equivalent relative to the chiral modifier.

The method may produce a product in at least about 90% chemical yield, optionally in essentially quantitative yield. It may produce a product with an enantiomeric excess of at least about 50%, optionally at least about 60%, and in at least about 90% chemical yield, optionally in essentially quantitative yield. It may do so across a wide range of substrates.

The nanoparticulate material may be recyclable. The method may comprise reusing the nanoparticulate reaction in a subsequent catalytic reduction. It may be recyclable multiple times without substantial loss of catalytic activity and/or of enantioselectivity (e.g. with loss of activity and/or of enantioselectivity between subsequent reactions of less than about 10%, or less than about 5, 2 or 1%).

In an embodiment there is provided a method for conducting a catalytic reduction comprising exposing a substrate to a catalytic nanoparticulate material according the first aspect, or made by the process of the second aspect, in the presence of a hydrogen source and a chiral modifier. The reduction may be at least partially enantioselective.

In another embodiment there is provided a method for conducting a catalytic reduction comprising exposing a substrate to a catalytic nanoparticulate material according the first aspect, or made by the process of the second aspect, in the presence of gaseous hydrogen at a pressure of less than about 750 kPa.

In another embodiment there is provided a method for conducting a catalytic reduction comprising exposing a substrate to a catalytic nanoparticulate material according the first aspect, or made by the process of the second aspect, in the presence of ammonium formate or alkaline isopropanol.

The invention also comprises a product, optionally an optically active product, made by the method of the third aspect.

In a fourth aspect of the invention there is provided use of a catalytic nanoparticulate substance according to the first aspect, or made by the process of the second aspect, in catalysis.

The catalysis may be catalysis of a hydrogenation reaction. It may be catalysis of an enantioselective reaction, e.g. of an enantioselective hydrogenation reaction.

### BRIEF DESCRIPTION OF THE DRAWINGS

A preferred embodiment of the present invention will now be described, by way of an example only, with reference to the accompanying drawings wherein:

FIG. 1 shows TEM (transmission electron microscope) images and (inset) selected area electron diffraction (SAED) of (A, B) FePt and (C, D) Pt nanowires.

FIG. 2 shows an EDX (energy-dispersive X-ray spectroscopy) analysis of (A) FePt and (B) Pt nanowires with Fe/Pt weight ratios of (A) 52:48 and (B) 5:95.

FIG. 3 is an XRD (X-ray diffraction) pattern of the as-synthesized (-: upper trace) FePt and ( - - - : lower trace) Pt nanowires.

FIG. 4 shows structures of some products of hydrogenation of activated ketones over 1 mol % of Pt nanowires.

FIG. 5 shows (A-C) TEM and (D) high-resolution TEM images of Pt nanowires (A) before use, (B) after 2 runs, and (C, D) after 10 runs.

FIG. 6 shows a proposed transition state model for the asymmetric hydrogenation of ethylpyruvate over alkaloid-modified Pt nanowires in water.

FIG. 7 is a photograph of reaction mixtures after the asymmetric hydrogenation of ethyl pyruvate over alkaloid-modified Pt nanowires in water. The catalyst and the ligand were dispersed in the aqueous phase, and the product was extracted into the solvent, ethyl acetate.

FIG. 8 shows TEM images of Pt nanorods synthesized with 150 mg of sodium oleate at 250° C.

FIG. 9 shows an EDX analysis of Pt nanorods.

FIG. 10 is a graph illustrating the effect of pressure on the (▪) conversion and () ee of asymmetric hydrogenation of ethyl pyruvate in water at 25° C. over 1 mol % of Pt nanowires.

FIG. 11 is a graph illustrating the effect of alkaloid concentration on the enantioselectivity of asymmetric hydrogenation of ethyl pyruvate in water at 25° C. over 1 mol % of Pt nanowires.

### DETAILED DESCRIPTION OF THE PREFERRED EMBODIMENTS

The present invention provides a nanoparticulate material comprising, optionally consisting of or consisting essentially of, metal nanowires. The nanoparticulate material may be suitable for use in catalysis. The nanowires may be straight or they may be bent. They may be nanorods. In this context, nanorods are considered to be straight nanowires.

The nanowires may be ultrathin. They may have a diameter of less than or equal to about 2 nm, or less than or equal to about 1.5 nm or less than or equal to about 1 micron. They may have a diameter of about 0.5 to about 2 nm, or about 0.5 to 1, 1 to 2, 1 to 1.5, 1.5 to 2 or 0.5 to 1.5 nm, e.g. about 0.5, 0.6, 0.7, 0.8, 0.9, 1, 1.1, 1.2, 1.3, 1.4, 1.5, 1.6, 1.7, 1.8, 1.9 or 2 nm. In the present specification, unless the context indicates otherwise, dimensions (diameter, length etc.) are expressed as mean dimension. Thus the diameter may be a mean diameter. The nanowires may have substantially constant diameter along their length. They may have a diameter that varies along its length by less than about 10% from the mean diameter, or less than about 5%. The extremely small diameter of the fibres provides a very high specific surface area. This is important in obtaining high catalytic activity. It should be noted that for a particular metal or mixture of metals, the specific surface area (i.e. surface area per unit mass) of the nanoparticulate material will increase linearly with a decrease in nanowire diameter. Thus the present nanowires, which have very small diameter and yet may be used unsupported, are particularly suited for catalytic purposes

The nanowires may have a length of greater than about 40 nm, or greater than about 50, 60, 70, 80, 90, 100, 150 or 200 nm, or they may be about 50 to about 500 nm, or about 50 to 200, 50 to 100, 100 to 500, 200 to 500, 100 to 200 or 100 to 150 nm, e.g. about 40, 50, 60, 70, 80, 90, 100, 110, 120, 130, 150, 150, 200, 250, 300, 350, 400, 450 or 500 nm. They may have a length greater than 500 nm, e.g. about 600, 700, 800, 900 or 1000 nm (or for example 50 to 2000 nm, 50 to 1000 nm, 100 to 2000 nm, 100 to 1000 nm or 500 to 1000 nm). This may be a mean length. In some cases, commonly those in which the nanowires are not straight, the nanowires may be from about 1 to about 20 microns in length, or about 1 to 10, 1 to 5, 1 to 2, 2 to 10, 5 to 10 or 2 to 5 microns, e.g. about 1, 1.5, 2, 2.5, 3, 3.5, 4, 4.5, 5, 5.5, 6, 6.5, 7, 7.5, 8, 8.5, 9, 9.5 or 10 microns or even longer. The nanowires may have an aspect ratio (i.e. length to diameter ratio) of at least about 25, or at least about 30, 35, 40, 45, 50, 55, 60, 65, 75, 80, 85, 90, 95 or 100, or of about 25 to about 250, or about 25 to 200, 25 to 150, 25 to 100, 25 to 50, 50 to 250, 100 to 250, 150 to 250, 35 to 250, 35 to 150, 35 to 100, 50 to 200, 50 to 150 or 50 to 100, e.g. about 25, 30, 35, 40, 45, 50, 55, 60, 65, 70, 75, 80, 85, 90, 95, 100, 110, 120, 130, 140, 150, 200 or 250. In some instances, particularly for very long nanowires of 1 to 20 microns as described above, the aspect ratio may be much higher than this. It may be for example about 250 to about 1000, or about 1000 to 10000, 1000 to 5000, 1000 to 2000, 2000 to 10000, 5000 to 10000 or 2000 to 5000, e.g. about 1000, 1500, 2000, 2500, 3000, 3500, 4000, 4500, 5000, 6000, 7000, 8000, 9000 or 10000. The nanowires may be unbranched nanowires. Suitable dimensions for the nanowires include, by way of example, mean diameter less than 2 nm and mean length greater than 40 nm, mean diameter less than 2 nm and mean length greater than 100 nm, mean diameter less than 1.5 nm and mean length greater than 40 nm, mean diameter less than 2 nm and mean length greater than 50 nm, zo mean diameter less than 1.5 nm and mean length greater than 50 nm, and mean diameter less than 1.5 nm and mean length greater than 100 nm. Each of these examples may be either straight or may be bent. Straight nanowires may have a mean diameter of less than about 2 nm and a length of about 50 to about 500 nm (or about 50 to 200 nm or about 100 to 500 nm). Bent or crooked nanowires may have a diameter of less than about 1.5 nm (or less than about 1 nm) and a length of greater than about 50 nm (or about 50 to about 1000 nm or about 100 to 1000 nm or about 1 to about 10 microns or about 1 to about 5 microns or about 5 to about 10 microns) or a diameter of less than about 1.5 nm and a length of about 1 to about 10 microns (or 1 to 5 or 5 to 10 microns). The nanowires may have predominant exposure of (111) planes on the surface thereof. The metal of the nanowires may be crystalline. Each nanowire may comprise (or consist essentially of) a single crystal. Thus the nanowires may be single crystal nanowires. This may be demonstrated for example by Transmission Electron Microscopy.

The nanowires of the present invention may be sufficiently robust that they do not require a support. They may be unsupported. They may be free-standing nanowires. They may be in the form of discrete nanowires, for example dispersed or suspended in a liquid. They may be in the form of a mat or wool or bed or mesh of nanofibres. It may be in the form of a precipitate. They may in some instances be supported on a support, e.g. on a carbon support. The nanowires may be used in a catalysed reaction in an unsupported form.

The metal of the metal nanowires may be a Group 8 to Group 11 element, or may be a mixture of any two or more (e.g. 2, 3, 4 or 5) Group 8 to Group 11 elements. In this context, Groups refer to groups in the periodic table, so that Group 8 to Group 11 includes Group 8 (the group including iron), Group 9 (the group including cobalt), Group 10 (the group including nickel) and Group 11 (the group including copper, sometimes referred to as Group Ib). The metal may be for example platinum, palladium, rhodium, ruthenium or gold or a mixture of any two or more of these. Thus the metal nanowires may comprise (or consist of or consist essentially of) an alloy or mixture of metals, e.g. of Group 8 to Group 11 metals. Other metals that may be used either alone or in combination with other metals include copper and iron. Particular examples of metals or combinations of metals include platinum, platinum/iron, iron/palladium, iron/ruthenium and platinum/ruthenium. The metal may be a mixture of platinum with at least one other Group 8 to Group 11 element, e.g. palladium, rhodium, ruthenium, iron or gold. Thus the nanowire may be a single metal nanowire or it may be a multimetal nanowire (e.g. a 2 metal, 3 metal, 4 metal or 5 metal nanowire). In the event that the metal nanowire is a single metal nanowire, the single metal may be at least about 90% pure on a mole or weight basis, or at least about 91, 92, 93, 94, 95, 96, 97, 98, 99, 99.5 or 99.9% pure, or may be about 91, 92, 93, 94, 95, 96, 97, 98, 99, 99.1, 99.2, 99.3, 99.4, 99.5, 99.6, 99.7, 99.8, 99.9 or 100% pure. The impurities, if present, may be metallic or may be non-metallic. In the event that the metal nanowire is a multimetal nanowire, the ratio between any two metals in the nanowire on a mole or weight basis may be about 1 to about 100 (i.e. about 1:1 to about 100:1) or about 1 to 50, 1 to 20, 1 to 10, 1 to 5, 1 to 2, 2 to 100, 5 to 100, 10 to 100, 20 to 100, 50 to 100, 2 to 50, 2 to 10, 10 to 50, 20 to 50 or 10 to 20, e.g. about 1, 1.5, 2, 2.5, 3, 3.5, 4, 4.5, 5, 6, 7, 8, 9, 10, 15, 20, 25, 30, 35, 40, 45, 50, 60, 70, 80, 90 or 100. For example the metal may be a mixture of platinum and iron, where the ratio of platinum to iron is about 10 to about 20, or where the ratio of iron to platinum is about 2 to about 3. The metal nanowires may have substantially no metal salt therein or thereon. They may have substantially no metal oxide therein. In this context, “substantially no” may allow for trace amounts derived for example from natural oxidation in air. It may indicate less than about 5% by weight or mole, or less than about 2, 1, 0.5, 0.2 or 0.1% by weight or mole.

In many embodiments of the invention the nanowires are not Fe/Pt nanowires. In other embodiments of the invention the nanowires are Fe/Pt nanowires in which the ratio of Pt to Fe is greater than about 1, or greater than about 2, 5, 10 or 20.

The invention therefore encompasses a nanoparticulate material comprising (optionally consisting essentially of or consisting of) long, ultrathin nanowires of platinum. It also encompasses a nanoparticulate material comprising (optionally consisting essentially of or consisting of) long, ultrathin nanowires of palladium, or of rhodium, or of ruthenium, or of gold, or of copper, or of iron, or of platinum/ruthenium. In some instances the nanoparticulate material may comprise more than one different type of long ultrathin metal nanowire, e.g. may comprise (or consist of or consist essentially of) nanowires of different metals and/or different combinations of metals. The invention also encompasses a nanoparticulate material comprising (optionally consisting essentially of or consisting of) long, ultrathin single crystal nanowires of platinum. It further encompasses a nanoparticulate material comprising (optionally consisting essentially of or consisting of) long, ultrathin single crystal nanowires of one or more Group 8 to Group 11 metals.

The nanoparticulate material may be catalytic. It may be catalytic for a reduction reaction. It may be catalytic for a hydrogenation reaction. The surface of the nanowires may be catalytically active. The nanowires may be single crystal nanowires. This feature promotes their catalytic activity, as does the high surface area that results from the very small diameter of the nanowires.

The metal nanowires may have a chiral modifier associated with them. In this context the term “associated” may indicate that the chiral modifier is adsorbed, e.g. chemisorbed, onto the surface of the nanowires. The chiral modifier may serve to direct a reaction catalysed by the nanoparticulate material to a particular optical isomer or diastereomer of product. The degree of direction (i.e. the optical purity of the product) may depend on the reaction conditions and on the nature of the chiral modifier. Typical values for enantiomeric excess of the product that may be obtained by use of an optically pure (e.g. greater than 95% optically pure, or greater than 96, 97, 98 or 99% optically pure) chiral modifier may be greater than about 50%, or greater than about 60, 70, 80 or 90%. Enantiomeric excess of about 50, 55, 60, 65, 70, 75, 80, 85, 90, 95, 96, 97, 98 or 99% may be achievable by selection of the appropriate reaction conditions (solvent, temperature, pressure, hydrogen source etc.). The chiral modifier may be any chiral species capable of directing the reaction to a particular optical isomer or diastereomer. It may be adsorbable onto the surface of the metal nanowires. It may be for example an alkaloid, or other chiral natural product, or may be an optically active aminoalcohol, an optically active diamine, an optically active phosphine or an optically active aminophosphine or may be a mixture of any two or more of these. Natural products such as alkaloids are convenient as chiral modifiers since they are commonly available in high optical purity from natural sources. Other naturally available chiral species such as chiral amino acids may also be used as chiral modifiers. Improved optical activity of a product to obtained using the nanowires as a catalyst is generally obtained when the chiral modifier is soluble in the reaction mixture. In cases where the reaction is conducted in an aqueous environment, it is therefore preferable that the chiral modifier be water soluble. In the case where the chiral modifier is basic (which is the case for many naturally occurring chiral materials such as alkaloids), it may therefore be preferable to solublise the chiral modifier by adding an acid, preferably at least about one mole equivalent relative to the chiral modifier, in order to protonate the chiral modifier. In this instance, the active chiral modifier will be the protonated form of the added chiral modifier, i.e. it may be for example a protonated alkaloid, a protonated aminoalcohol etc. The chiral modifier may be associated with the nanowires in situ, i.e. in the process of conducting a catalysed reaction using the nanowires, or it may be associated with the nanowires in a separate step prior to conducting the catalysed reaction.

The nanoparticulate material may be made by exposing a mixture, optionally a homogenous solution, of a suitable precursor and an amine to a metal carbonyl at elevated temperature. The precursor should be soluble in an organic solvent.

The precursor may comprise the metal or mixture of metals present in the metal nanowires, e.g. if the nanowires are platinum nanowires, the precursor may comprise a platinum compound, and if the nanowires are platinum/iron nanowires, the precursor may comprise a platinum compound and an iron compound or a platinum/iron compound. This is not necessarily the case however. For example, in making platinum/iron nanowires, a platinum precursor may be used and the iron may be provided by use of iron pentacarbonyl, which may also function as a reducing agent. The precursor should comprise at least one of the metals present in the nanowires to be produced. If the nanowires are single metal nanowires, the precursor should comprise the metal of the nanowires. The precursor or, in the event that the more than one precursor is used, at least one of the precursors (or each independently) may be a metal complex or a metal compound. It may be a reducible metal complex or metal compound. It may be a metal complex or metal compound which is reducible to the metal. The complex may be for example an acetylacetone (acac) complex. The precursor may be a metal salt of an organic acid, e.g. of a long chain organic acid (for example C12 to C18 organic acid). Suitable metal salts include for example oleate.

The amine may be a C6 to C20 amine, or C6 to C12, C12 to C20 or C16 to C20, e.g. C6, C7, C8, C9, C10, C11, C12, C13, C14, C15, C16, C17, C18, C19 or C20. It may be a primary amine. It may be linear. It may be branched. It may be cyclic. It may be unsaturated. It may be an alkenylamine or may be an alkynylamine or may comprise both double and triple bonds. The amine may function as a solvent. The mixture may comprise no solvent other than the amine. The mixture may be a solution. In forming the mixture, it may be necessary to heat the amine and the precursor. Suitable temperatures are commonly about 50 to about 150° C., or about 50 to 100, 100 to 150, 60 to 120 or 80 to 130° C., e.g. about 50, 60, 70, 80, 90, 100, 110, 120, 130, 140 or 150° C. The mixture may be degassed and/or flushed with an inert gas before, during or after the formation of the mixture. Suitable inert atmospheres include nitrogen, neon, helium, argon, carbon dioxide and mixtures thereof. The inert atmosphere should be a non-oxidising atmosphere. It should be a substantially anoxic atmosphere.

The step of exposing the mixture of the precursor and the amine to a metal carbonyl may be conducted under an inert atmosphere or a non-oxidising atmosphere, e.g. a noble gas or other gas as described above.

The temperature used for reducing the precursor may be between about 100 and about 300° C., or about 150 to 300, 200 to 300, 100 to 200 or 150 to 200° C., e.g. about 100, 110, 120, 130, 140, 150, 160, 170, 180, 190, 200, 210, 220, 230, 240, 250, 260, 270, 280, 290 or 300° C.

In some instances, metal nanowires are required that do not comprise the metal of the metal carbonyl. In such instances, the process may optionally comprise an additional step of treating the initially formed nanowires with an etchant so as to remove the unwanted metal. The etchant should therefore be, or comprise, a substance capable of solublising the metal of the metal carbonyl but not capable of solublising the metal of the precursor to an appreciable degree. A commonly used etchant is an acid, since this will readily dissolve the iron of iron pentacarbonyl, a useful metal carbonyl for the present process, and will essentially not dissolve many of the other metals that may be may required in the nanowires, such as gold, platinum, palladium etc. The acid may be a mineral acid. It may be a hydrohalic acid such as hydrochloric acid. It is commonly used in a concentration sufficient to achieve an acceptable rate of dissolution of the metal to be dissolved. Suitable concentrations are about 2 to about 2M, or about 2 to 5, 5 to 10 or 3 to 7M, e.g. about 2, 3, 4, 5, 6, 7, 8, 9 or 10M. These may be aqueous or may be in some short chain alcohol such as methanol or ethanol. The use of an etchant to remove unwanted metal may result in particularly thin nanowires, e.g. under about 1.5 nm or even under 1 nm in diameter. Thus in some embodiments of the invention, the nanowires may be made by a process that comprises preparing mixed metal nanowires, optionally by known methods, and then etching out one or more unwanted metals from the nanowires.

The process for preparing the nanowires may comprise controlling the length of the nanowires. It may comprise adding a length control agent. The length of the nanowires may in some instances be controlled by addition of a suitable length control solvent. An example of a suitable length control solvent is ODE (1-octadecene). The length control solvent may be an alkene. It may be a C12 to C20 alkene, or a C16 to C20 alkene. It may be straight chain or may be branched. It may be a terminal alkene or a non-terminal alkene. It may comprise one or more alicyclic rings and/or aromatic rings. It may be a mixture of any two or more such suitable solvents. More generally, the length control solvent should be a high temperature solvent (i.e. it should not decompose or break down under high temperatures such as those used in the reaction to make the nanowires). The length control solvent may be a non-coordinating solvent. It may not coordinate with the metal of the nanowires. It may be compatible and/or miscible with the amine used in making the nanowires. The ratio of the length control solvent to the amine may be about 0.2 to 5 (i.e. 1:5 to 5:1) on a weight, volume or mole basis, or about 0.2 to 3, 0.2 to 1, 0.2 to 0.5, 0.5 to 5, 1 to 5, 2 to 5, 0.5 to 2 or 1 to 3, e.g. about 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5 or 5. In a specific example the length could be controlled by adding ODE to a solution of oleylamine and the precursor before addition of the metal carbonyl. In this case, when the ratio of oleylamine to ODE was 1:1 by volume, the length of the resulting nanowires was about 500 nm, and when the ratio was 1:3, the length was 20 nm. By contrast, in the absence of ODE, the nanowires were more than 10 microns in length. Thus the length control solvent may be viewed as a length shortening solvent.

Thus a suitable process for making the nanoparticulate material comprises:

a) preparing a mixture of a precursor (e.g. Pt(acac)₃) and an amine (e.g. oleylamine) together with a length control solvent (e.g. ODE), said precursor being capable of being converted to a metal or a mixture of metals;
b) exposing the mixture to a metal carbonyl (e.g. Fe(CO)₅) at elevated temperature to form nanowires; and
c) optionally treating the nanowires with an etchant capable of removing the metal of the metal carbonyl;
so as to produce the nanoparticulate material in the form of metal nanowires. In this process, the ratio of amine to length control solvent is commonly in the range of about 2:1 to about 1:3 on a volume.

The ratio of the precursor to the metal carbonyl may be about 1 to about 500% based on moles of metal, or about 1 to 400, 1 to 300, 1 to 200, 1 to 100, 1 to 50, 1 to 20, 1 to 10, 1 to 5, 5 to 500, 10 to 500, 20 to 500, 50 to 500, 100 to 500, 200 to 500, 5 to 200, 5 to 100, 5 to 50, 10 to 200, 10 to 100, 10 to 50, 50 to 100, 100 to 200, 200 to 300, 300 to 500 or 200 to 400%, e.g. about 1, 2, 3, 4, 5, 6, 7, 8, 9, 10, 15, 20, 25, 30, 35, 40, 45, 50, 60, 70, 80, 90, 100, 110, 120, 130, 140, 150, 200, 250, 300, 350, 400, 450 or 500%. In cases where the ratio of precursor to metal carbonyl is substantial, e.g. over about 10% on a mole basis, it may be beneficial to etch out the metal of the metal carbonyl as described above. In cases in which the metal carbonyl is used in very minor amounts, e.g. less than about 10%, it may be simpler to leave the metal of the metal carbonyl in place. The metal carbonyl may be used in trace amounts relative to the metal of the precursor, e.g. less than about 10% on a weight or mole basis, or less than about 9, 8, 7, 6, 5, 4, 3, 2, 1, 0.5, 0.2 or 0.1%.

The mixture of the precursor and the amine may also comprise a carboxylic acid salt. This may result in production of metal nanorods, i.e. straight nanowires. The carboxylic acid may be a C6 to C18 carboxylic acid salt or C6 to C12, C12 to C20 or C16 to C20, e.g. C6, C7, C8, C9, C10, C11, C12, C13, C14, C15, C16, C17, C18, C19 or C20. It may be linear. It may be branched. It may be cyclic. It may be unsaturated. It may be an alkenoic acid salt or may be an alkynoic acid salt or may comprise both double and triple bonds. The hydrocarbon chain of the carboxylic acid salt may be the same as that of the amine, or may be different. The carboxylate salt may be, or may function as, a surfactant. The salt may be Group 1 metal salt, e.g. a sodium or potassium salt. In one embodiment, the amine is oleylamine and the salt is sodium oleate. Thus in some embodiments the nanowires may be made by exposing a precursor to a small amount (on a molar basis) of a metal carbonyl in the presence of an amine and a carboxylate salt. These embodiments may provide straight nanowires, i.e. nanorods.

The process may additionally comprise exposing the metal nanowires to a chiral modifier. Suitable chiral modifiers have been described above. The step of exposing the metal nanowires to the chiral modifier so as to associate the chiral modifier with the nanowires may be conducted as a discrete step or it may be conducted in situ as part of the method of conducting a chirally directed reaction using the nanoparticulate material. Suitable solvents and conditions for this are the same as for conducting reactions with the nanoparticulate material, as described below.

The nanoparticulate material of the present invention may be used for conducting a catalytic reaction, e.g. a catalytic reduction. Thus exposure of a substrate to the catalytic nanoparticulate material in the presence of a hydrogen source may lead to reduction of the substrate. In this context, the term “hydrogen source” refers to a source of the element hydrogen and may not refer necessarily to a source of molecular hydrogen. It may for example refer to a source of hydrogen atoms.

In the reactions described herein using the nanoparticulate material as catalyst, the nanowires of the nanoparticulate material may be unsupported. It may be used unsupported in a catalysis reaction. This may serve to distinguish them from supported catalysts, such as platinum on carbon, platinum on metal oxide etc.

The reduction may be at least partially stereospecific or enantiospecific in the event that a chiral modifier is used. As discussed above, the chiral modifier may be associated with the metal nanowires of the nanoparticular material in a discrete step, or may be added to the reaction mixture for conducting the catalytic reduction. The chiral modifier may be used in an amount approximately equal to that of the substrate on a molar basis. The ratio of chiral modifier to substrate on a molar basis may be about 0.5 to about 2 (i.e. about 1:2 to about 2:1), or about 0.5 to 1, 1 to 2, 1 to 1.5 or 1.5 to 2, e.g. about 0.5, 0.6, 0.7, 0.8, 0.9, 1, 1.1, 1.2, 1.3, 1.4, 1.5, 1.6, 1.7, 1.8, 1.9 or 2. The ratio of metal nanowires to chiral modifier may be about 1 to about 100 (i.e. about 1:1 to about 100 to 1) on a weight basis, or about 1 to 50, 1 to 20, 1 to 10, 1 to 5, 1 to 2, 2 to 100, 5 to 100, 10 to 100, to 100, 50 to 100, 2 to 50, 5 to 50, 10 to 50, 20 to 50, 5 to 20, 5 to 10, 10 to 20 or 5 to 15, e.g. about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90 or 100.

The nanowires may be used in a ratio to the substrate of about 0.01 to about 10% by weight or mole, or about 0.02 to 10, 0.05 to 10, 0.1 to 10, 0.2 to 10, 0.5 to 10, 1 to 10, 2 to 10, 5 to 10, 0.1 to 5, 0.1 to 2, 0.1 to 1, 0.1 to 0.5, 0.5 to 5, 1 to 5, 2 to 5, 1 to 2, 0.5 to 1 or 0.5 to 2%, e.g. about 0.01, 0.02, 0.03, 0.05, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 6, 7, 8, 9 or 10% on a mole or weight basis.

A suitable combination for the reactions of the present invention is about 0.5 to 2 mol % Pt-nanowire catalyst, with a chiral modifier at about 5:1 to about 20:1 Pt-to-chiral modifier weight ratio, e.g. about 1 mol % Pt-nanowire catalyst with Pt-to-alkaloid weight ratio of about 10:1.

Particular examples of metal nanowires which may be used in the catalytic reduction include platinum nanowires, platinum/ruthenium nanowires and platinum/iron nanowires.

The reaction may be conducted in any suitable solvent, for example alcohols, hydrocarbons (e.g. aromatic hydrocarbons), halogenated solvents, organic acids, dipolar aprotic solvents, protic solvents or mixtures of any two or more of these. In some instances the reaction may be conducted in the absence of solvent (i.e. neat). Suitable solvents include methanol, ethanol, toluene, dichloromethane, acetic acid, tetrahydrofuran, t-butanol, 2-propanol, acetone or water/acetic acid (1:1). The reaction may be conducted in an aqueous medium, e.g. in water (optionally water unmixed with any organic solvent other than, if needed, an organic acid for protonation of a chiral modifier). This, together with the recyclability of the nanowire catalyst, contributes to the environmentally friendly or “green” nature of the reaction. The substrate may be in solution in the solvent (if present) or may be not in solution or may be partially in solution. The inventors have observed that in the event that a chiral modifier is used, the enantiomeric excess may be dependent on the nature of the solvent. Solvents comprising organic acids, e.g. water soluble organic acids, may be used. For example suitable solvents include acetic acid and aqueous acetic acid. The proportion of organic (e.g. acetic acid) in the aqueous acid may be about 0.1 to about 99.9%, or about 0.1 to 90, 0.1 to 50, 0.1 to 20, 0.1 to 10, 0.1 to 5, 0.1 to 2, 0.1 to 1, 0.1 to 0.5, 0.1 to 0.2, 0.2 to 99.9, 1 to 99.9, 2 to 99.9, 5 to 99.9, 10 to 99.9, 20 to 99.9, 50 to 99.9, 80 to 99.9, 90 to 99.9, 99 to 99.9, 1 to 50, 50 to 90, 99 to 99, 1 to 10, 10 to 50, 20 to 50 or 50 to 70% on a weight or volume basis, e.g. about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 91, 92, 93, 94, 95, 96, 97, 98, 99, 99.1, 99.2, 99.3, 99.4, 99.5, 99.6, 99.7, 99.8 or 99.9%. The amount of organic acid may be sufficient to completely protonate the chiral modifier. This is useful in the case where the chiral modifier itself has low solubility in water and the reaction is conducted in an aqueous medium. Thus the ratio of organic acid to chiral modifier may be at least about 1:1, and may be at least about 1.5:1 or 2:1, or may be 1:1 to about 10:1 or 1:1 to about 5:1 or about 1:1 to about 2:1, or about 1:1 to about 1.5 to 1, e.g. about 1:1, 1.1:1, 1.2:1, 1.3:1, 1.4:1, 1.5:1, 2:1, 2.5:1, 3:1, 3.5:1, 4:1, 4.5:1 or 5:1 or may be greater than 5:1. The organic acid may be sufficiently strong an acid to be capable of protonating the chiral modifier.

The reaction may be conducted at a temperature of about room temperature, or about 15 to about 30° C., or about 15 to 25, 20 to 30 or 20 to 25° C., e.g. about 15, 20, 25 or 30° C. In some instances the reaction may be conducted under an inert or non-oxidising atmosphere. It may be conducted under a reducing atmosphere. Suitable atmospheres include hydrogen, nitrogen, neon, helium, argon, carbon dioxide and mixtures thereof.

The hydrogen source may be hydrogen gas. The hydrogen gas may be at a pressure of less than about 750 kPa, or less than about 700, 600, 500, 400, 300 or 100 kPa, or at a pressure of about 100 to about 750 kPa, or of about 200 to 750, 400 to 750, 500 to 750, 600 to 750, 100 to 500, 100 to 300, 500 to 700, 500 to 600 or 600 to 700 kPa, e.g. about 100, 150, 200, 250, 300, 350, 400, 450, 500, 550, 600, 650, 700 or 750 kPa. These relatively low pressures render the reaction convenient as they do not require equipment capable of dealing with very high pressures.

The time required for the reaction may depend on the reaction conditions, e.g. the source of hydrogen, the pressure of hydrogen gas (if used) or the concentration of the source of hydrogen, the ratio of substrate to catalyst, the temperature etc. Typical times are from about 1 to about 10 hours, for example about 1 to 5, 5 to 10 or 5 to 7 hours, e.g. about 1, 2, 3, 4, 5, 6, 7, 8, 9 or 10 hours.

Other hydrogen sources which may be used include ammonium formate and secondary alcohols. Suitable secondary alcohols include isopropanol, isobutanol, 2-phenyl-2-propanol etc. Secondary alcohols may be used in conjunction with an alkaline salt such as sodium hydroxide or potassium hydroxide.

Suitable substrates that may be reduced using the reaction described above include α-ketoesters, a-ketolactones, a-iminoesters, a-ketoaryl or a-ketoheteroaryl compounds (e.g. alkyl phenyl ketones) etc. The nanoparticulate materials of the present invention may also be used to catalyse carbenoid insertion reactions, for example the reaction of an alkene with an azido compound to produce a cylclopropane. Suitable alkenes include arylalkenes (styrenes), heteroarylalkenes etc. Other reactions using the nanoparticulate materials of the invention as catalysts include selective hydrogenation of acetylenes to olefins, hydrosilylation and hydrogenative aldol coupling.

In the event that an optical modifier is used, the resulting optically active product may have an enantiomeric excess of at least about 50%, or at least about 60, 70, 80 or 90%, or about 50 to about 90%, or about 50 to 70, 70 to 90, 60 to 80 or 80 to 90%, e.g. about 50, 55, 60, 65, 70, 75, 80, 85, 90, 95 or 100%.

The method may produce a product in at least about 90% chemical yield, or at least about 95 or 99% yield, e.g. about 90, 91, 92, 93, 94, 95, 96, 97, 98, 99, 99.5 or 100% yield.

The nanoparticulate material may be recyclable, i.e. it may be reused in a subsequent catalytic reaction. It may be reused in at least 2, 3, 4, 5, 6, 7, 8, 9 or 10 successive reactions without substantial loss of activity. It may be reused this number of times without loss of activity (as gauged by % yield of product and/or by enantioselectivity) of greater than about 20%, or about 15, 10, 5, 2 or 1%. When using a chiral modifier, it may be necessary to add further chiral modifier when reusing the nanoparticulate material. In some instances the chiral modifier may remain associated with the nanowires when isolating the nanoparticulate material from a reaction mixture. There may be no need to add further chiral modifier when reusing the nanoparticulate material. In reusing the nanoparticulate material, it may simply be removed from the reaction mixture, e.g. by filtration, microfiltration, centrifuging/decanting etc., optionally washed with a solvent to remove residual reaction mixture, and then reused in a subsequent reaction.

A totally green process for enantioselective hydrogenation of a-ketoesters over Pt nanowires is described herein. Platinum nanowires with a diameter of 1-2 nm were successfully synthesized and used for the asymmetric hydrogenation of a-ketoesters in the presence of cinchona alkaloids under a low pressure at room temperature in water, giving quantitative yields and excellent enantioselectivity of up to 72-94%. The catalyst along with the modifier was recycled multiple times without any significant loss in activity and selectivity. Such a catalyst system is unprecedented for asymmetric hydrogenation of ketoesters.

Thus the present invention describes a simple and totally green approach to the synthesis of uniform nanostructures of platinum and other metals, e.g. nanowires, nanorods, nanoparticles and nanocomposites, and their applications as catalysts for organic reactions, especially asymmetric hydrogenation. The materials may be used for chiral and non-chiral organic reactions, giving excellent yields and selectivity of the products. The nanostructures are highly stable, and the reactions can be conducted under green conditions using water as the solvent. The product isolation may be performed by extracting the product from water using an organic solvent, whereby the catalyst system remains in the aqueous phase. This novel catalyst can be recycled several times without significant loss in activity and selectivity. For example, in the case of catalytic hydrogenation of ethyl pyruvate, the catalyst was recycled 10 times without any significant loss in activity and selectivity. Hydrogenation is successfully catalyzed at comparatively low pressure, at room temperature and in water, giving quantitative conversions and enantioselectivities ranging from 72% to 94%. The catalyst may also be effectively employed in various other reactions, such as selective hydrogenation of acetylenes to olefins, hydrosilylation and hydrogenative aldol coupling. The metal nanostructures described and produced herein have potential as green catalysts, in the pharmaceuticals and specialty chemicals industries.

Nanostructures and nanocomposites of transition metals are of great interest in the development of green chemical processes, such as hydrogenation, carbonylation, hydroformylation, coupling reactions, and multicomponent reactions. Platinum nanowires (1-2 nm in diameter and 100 nm in length) demonstrate interesting characteristics as heterogeneous catalysts for pharmaceuticals synthesis. Other metal nanostructures show excellent selectivity in various other reactions. These catalysts are of interest for industrial applications.

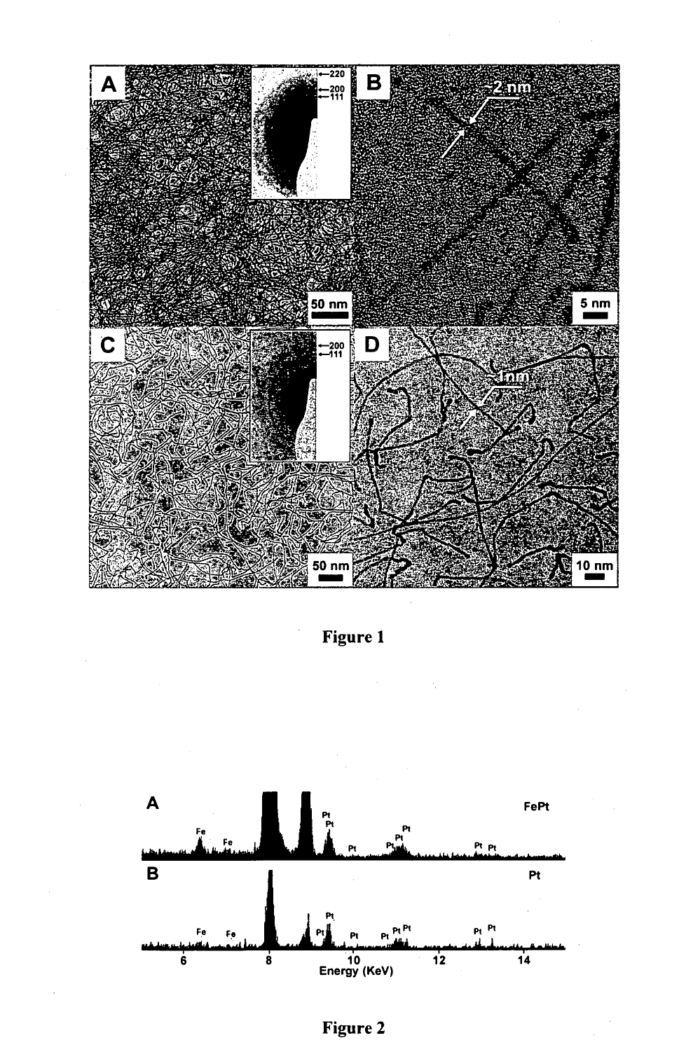

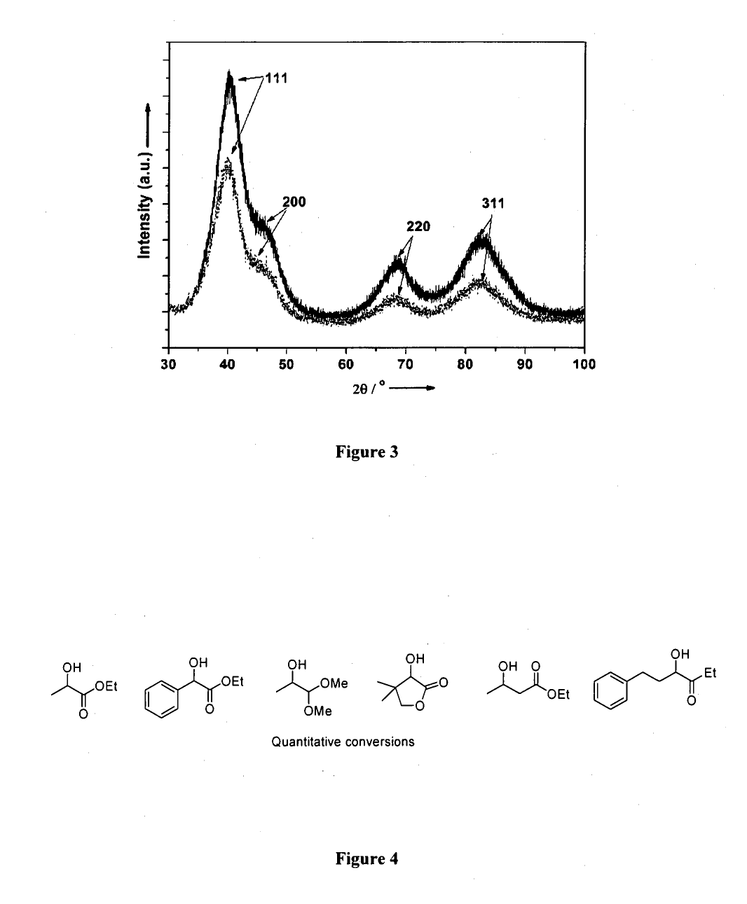

The inventors have synthesised novel platinum nanowires and nanorods with uniform length and a diameter of around, or less than, about 1 nm. The nanowires and nanorods were characterized in detail by transmission electron microscopy (TEM) (FIG. 1), energy dispersive X-ray (EDX) analysis (FIG. 2) and X-ray diffraction (XRD) (FIG. 3). These nanowires and nanorods exhibit unique properties, for example, they have over preferential exposure of (111) planes on the surface (FIG. 3). Due to the high interest in asymmetric hydrogenation of activated ketoesters, the nanowires and nanorods were examined as novel catalysts to hydrogenate these substrates (Scheme 3).

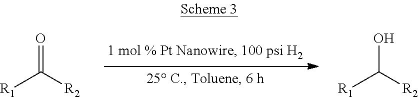

In initial studies, the inventors focused on the racemic hydrogenation of ethyl pyruvate. Quantitative conversion to ethyl lactate was achieved under a hydrogen pressure of 100 psi (about 690 kPa) in 6 h with 1 mol % of nanowire or nanorod catalyst (Scheme 3). Hydrogenation of other activated ketones also proceeded well, producing the corresponding alcohols in quantitative yield (FIG. 4).

The inventors also performed an asymmetric version of the hydrogenation of ethyl pyruvate in the presence of cinchona alkaloids. Without wishing to be bound by theory, the inventors hypothesise a proposed catalytic cycle consisting of a fast adsorption of ketone and hydrogen on the Pt surface, stepwise addition of the two adsorbed hydrogen atoms to the C═O bond with the half hydrogenated intermediate, followed by the fast desorption of the alcohol. The modified catalyst was not only enantioselective, but also much more active than the unmodified one due to ligand acceleration. The effects of catalyst loading, alkaloid and substrate concentrations, hydrogen pressure, solvents and temperature were investigated.

The best result was obtained using 1 mol % of the catalyst with a Pt-to-alkaloid weight ratio of 10:1 in toluene at 25° C. at a hydrogen pressure of 100 psi (Scheme 4).

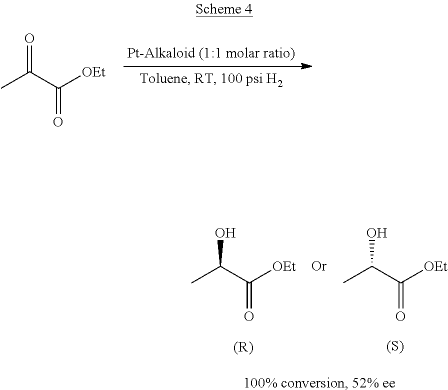

Cinchonidine (Cd), dihydrocinchonidine (HCd), quinidine (Qd) and dihydroquinidine (HQd) gave (R)-alcohols, whereas cinchonine (Cn), dihydrocinchonine (HCn), quinine (Qn) and dihydroquinine (HQn) gave (S)-alcohols in nearly quantitative yields and 72-94% enantiomeric excess (ee). The reaction was highly solvent- and concentration-dependent. Reaction in toluene or ethanol resulted in the best yield and enantioselectivity. Slight improvement in enantioselectivity (by 2-3% ee) was also achieved under a low pressure of 40 psi (about 275 kPa).

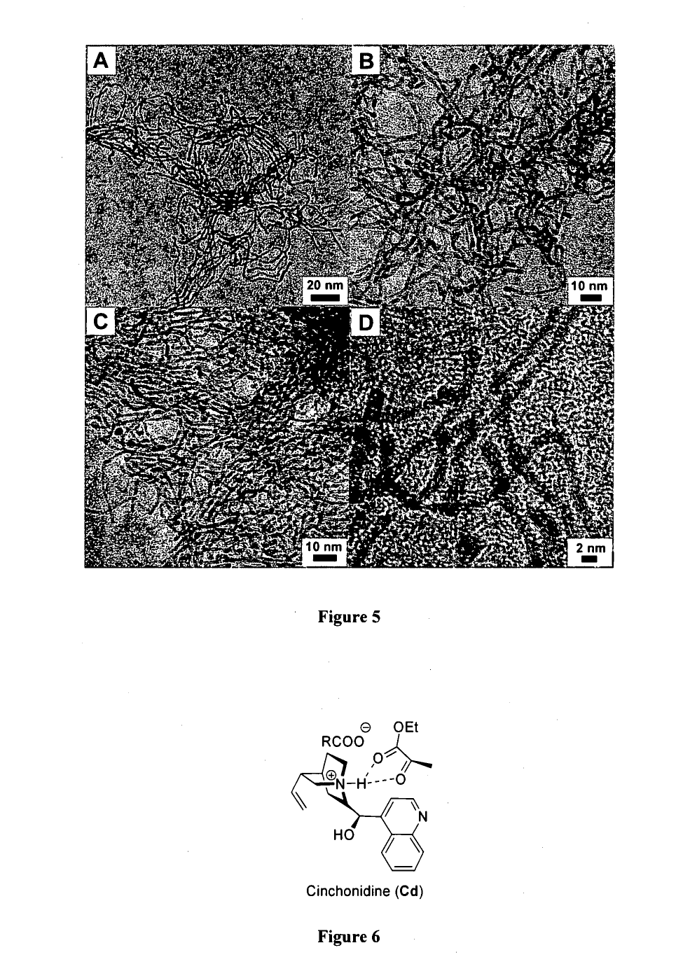

Reactivity and selectivity varied slightly with alkaloid-to-Pt ratio. The maximum rate and selectivity or complete modification was achieved at lower modifier concentrations, suggesting that the product-determining interactions between modifier and substrate for enantiodifferentiation occurred on the surface and not in solution. Moisture and air did not affect rate and selectivity noticeably. This is evidenced by the fact that the reactions worked well in aqueous conditions and the reagents could be weighed and transferred under an air atmosphere with little observed adverse effect on the reaction. The catalyst was recovered by simple centrifugation, and was recycled 10 times in ethyl pyruvate hydrogenation without significant loss in activity and selectivity. The nanowire catalyst remained intact after 10 recycles, with no noticeable change in structure (FIG. 5).

The effect of various solvents as reaction medium was examined. Table 1 shows that acetic acid and methanol led to the best enantioselectivity.

TABLE 1
Solvent effects on the asymmetric hydrogenation of ethylpyruvate
over alkaloid-modified platinum nanowires.^{a}

| Entry | Solvent | Conversion (%)^{b} | ee (%)^{c} |
|---|---|---|---|
| 1 | Neat | 4 | 23 |
| 2 | Methanol | 100 | 66 |
| 3 | Ethanol | 100 | 40 |
| 4 | Toluene | 100 | 52 |
| 5 | Dichloromethane | 72 | 18 |
| 6 | Acetic acid | 100 | 72 |
| 7 | Tetrahydrofuran | 80 | 35 |
| 8 | t-Butanol | 92 | 40 |
| 9 | 2-Propanol | 96 | 42 |
| 10 | Acetone | 88 | 30 |
| 11 | Water | 15 | 45 |
| 12 | Acetic acid/water (1:1) | 100 | 74 |

^{a}The hydrogenation reactions were performed at room temperature under 100 psi (about 690 kPa) of hydrogen using a catalyst-to-ligand (cinchonine) molar ratio of 1:1.
^{b}Determined by HPLC analysis.
^{c}The ee (enantiomeric excess) and absolute configuration were determined by chiral HPLC analysis. The absolute configuration of the alcohol product was confirmed as (S) when cinchonine was used as the ligand by comparison to an authentic sample of the alcohol or by comparison of the sign of the optical rotation with literature data.

The reaction did not proceed well in pure water since the modifier was not soluble in water (Table 1, entry 11). However, a 1:1 mixture by volume of acetic acid and water provided quantitative conversion and 74% ee. The effect of various acids on the enantioselectivity of the reaction was then investigated. Various mineral acids and organic carboxylic acids were studied as a mixture with water. A minimum amount of 1:1 molar ratio of alkaloid and carboxylic acid, forming all the tertiary quinuclidine nitrogen to quaternary quinuclidine nitrogen, was necessary to give consistent conversion and selectivity (Table 1, Entry 11 versus Table 2). One equivalent of acetic acid is necessary to completely protonate the alkaloid to form a quarternary center. The quarternary salt is highly soluble in water and the reaction happens very smoothly. In water and in the absence of acid, the alkaloid remain insoluble and is suspended in the reaction mixture. In this case the interaction with platinum surface is minimal. 1% of acetic acid still provides many equivalents of the alkaloid used so that with 1% acid all the quinuclidine nitrogens will be protonated.

TABLE 2
Effects of various acids on the asymmetric hydrogenation of
ethylpyruvate over alkaloid-modified platinum nanowires.^{a}

| Entry | Acid^{b} | Conversion (%)^{c} | ee (%)^{d} |
|---|---|---|---|
| 1 | Formic acid | 65 | 32 |
| 2 | Acetic acid | 100 | 72 |
| 3 | Trifluoroacetic acid | 100 | 55 |
| 4 | Methane sulfonic acid | 100 | 15 |
| 5 | Citric acid | 100 | 58 |
| 6 | Malic acid | 100 | 58 |
| 7 | Camphoric acid | 10 | 3 |
| 8 | Benzoic acid | 15 | 5 |
| 9 | Acetic acid/water (1:1) | 100 | 74 |
| 10 | Acetic acid/water (1:3) | 100 | 72 |
| 11 | Acetic acid/water (1:9) | 100 | 72 |
| 12 | Acetic acid/water (1:19) | 100 | 70 |
| 13 | Acetic acid/water (1:99) | 100 | 74 |
| 14 | Acetic acid/water (0.1:99.9) | 100 | 71 |

^{a}The hydrogenation reactions were performed at room temperature under 100 psi (about 690 kPa) of hydrogen using a catalyst-to-ligand (cinchonine) molar ratio of 1:1.
^{b}Acids were used in 1:1 molar ratio with respect to alkaloid (Entries 1-8).
^{c}Determined by HPLC analysis.
^{d}The ee and absolute configuration was determined by chiral HPLC analysis. The absolute configuration of the alcohol product was confirmed as (S) when cinchonine was used as the ligand by comparison to an authentic sample of the alcohol or by comparison of the sign of the optical rotation with literature data.

Based on the experimental data, the verified structure and conformation of cinchona alkaloids, as well as the widely accepted adsorption model, the structure of the intermediate responsible for the enantioselectivity was proposed (see FIG. 6). In water, the intermediate was generated via the interaction of the protonated quinuclidine ring (which acted as an electrophilic agent) with the nucleophilic oxygen atom of the keto group of the ketoesters.

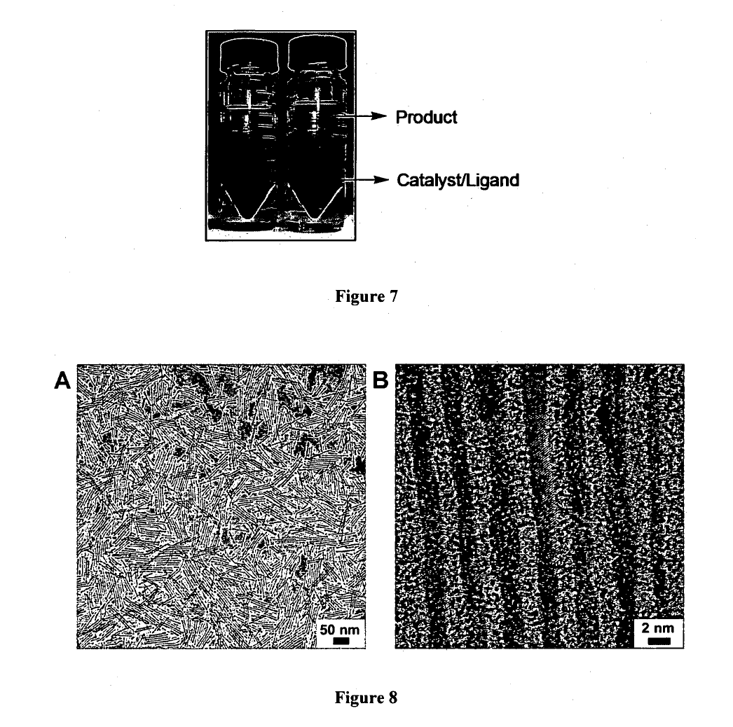

The nanowire catalyst along with modifier ligand salt was recycled 10 times without any significant loss in activity and selectivity (FIG. 7). The recycling was conducted by simply adding an organic solvent, and the product was isolated in the organic phase. The chiral modifier is a salt after protonation with acetic acid and the salt is highly soluble in water. The salt form of modifier is not commonly extracted by normal organic solvents and there was therefore no need to add further alkaloid when recycling the catalyst. It was found that different batches of Pt nanowires all resulted in quantitative yield in the asymmetric hydrogenation of ethylpyruvate, but the enantioselectivity varied substantially from 65% to 94%. The results indicated that the surface chemistry and interactions between the ligands, Pt wires and substrates were critical in these reactions.

Using a similar approach for the hydrogenation of methyl benzoylformate, the corresponding methyl mandelates were obtained in quantitative conversions and enantioselectivities ranging from 44% to 51% (Scheme 5).

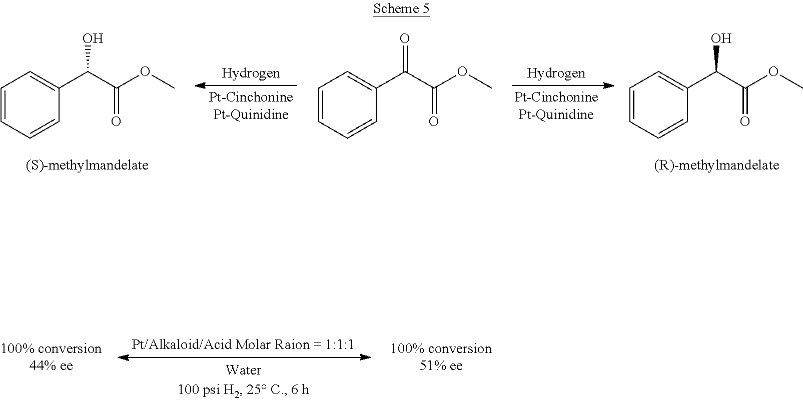

This is thought to be the first use of Pt nanowires and nanorods in the enantioselective hydrogenation of β-ketoesters. The Pt nanowire and nanorod catalysts exhibited superior reactivity and selectivity due to their unique surface properties.

In summary, the inventors have successfully synthesized uniform platinum nanowires and nanorods. These novel platinum nanostructures were employed as an effective heterogeneous catalyst for the asymmetric hydrogenation of ketoesters. They demonstrated excellent yields and moderate-to-excellent enantioselectivities. The catalysts were stable under moisture and air, and allowed for the first asymmetric hydrogenation of ketoesters in water. The reactions proceeded well at room temperature and a low hydrogen pressure. The catalysts were easily recycled by phase separation whereby the ligands and catalysts remained in the aqueous phase. They were stable to usage under normal atmospheric conditions, and were recycled under green conditions that are attractive for industrial processes.

### Examples

### Materials

The synthesis of Pt nanorods and nanowires were performed using commercially available reagents: platinum (II) acetylacetonate (Sigma-Aldrich, 97%), oleylamine (Sigma-Aldrich, >70%), iron pentacarbonyl (Sigma-Aldrich, 99.999%), concentrated hydrochloric acid (fuming) (Merck, 37%). Ethyl pyruvate, acetic acid and cinchona alkaloids were obtained from Sigma-Aldrich.

Synthesis of FePt Nanowires 200 mg of Pt(acac)₂ were mixed with 20 ml of oleylamine. The mixture was degassed by bubbling argon at 60° C. for 5 min. Temperature of the mixture was increased to 120° C. in 5 min to ensure a clear yellow solution. Upon heating at 120° C. for 30 min, 0.15 ml Fe(CO)s was injected into the hot solution, which darkened in color rapidly. The temperature was gradually raised to 160° C. and maintained for 30 minutes. The reaction was then cooled to room temperature and centrifuged in excess isopropanol. The supernatant was discarded, and the collected precipitate was redispersed in toluene. Further separation was performed by adding ethanol and centrifuging at high speed (5000 rpm).

### Synthesis of Pt Nanowires

Ultrathin Pt nanowires were achieved by an acidic etching method. HCl/methanol solution (5 M) was added to the as-prepared FePt nanowire precipitates. After 20 minutes of sonication, black precipitates were obtained following 10 minutes of centrifugation (3000 rpm); the yellowish green solution was discarded. The precipitates were subjected to another acidic treatment, and the dark solid was washed with pure methanol twice.

Synthesis of Pt Nanorods with Sodium Oleate

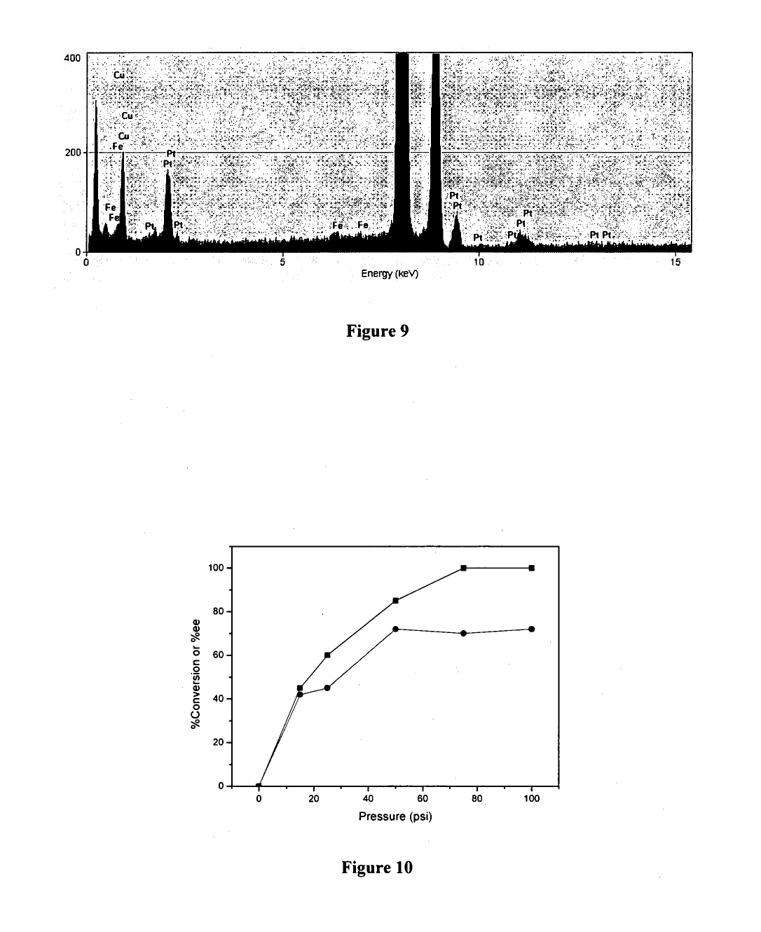

200 mg of Pt(acac)₂ and 150 mg of sodium oleate were added to 20 ml of oleylamine. The reaction mixture was degassed at 120° C. by bubbling argon for 15 min. As the solution turned clear yellow, a drop of Fe(CO)ₛ (about 0.005 ml) was injected into the hot solution. The solution turned dark in colour rapidly. The temperature was increased to 250° C. and maintained for 30 min. The reaction was then cooled to room temperature, and the sample was centrifuged in excess isopropanol. The supernatant was discarded, and the precipitates collected were redispersed in toluene. Further separation was conducted by adding ethanol and centrifuging at high speed. An electron micrograph of the resulting nanorods is shown in FIG. 8, and an EDX of the nanorods is shown in FIG. 9.

Catalytic hydrogenation of ethyl pyruvate was also performed over Pt nanorods. Quantitative conversions to the corresponding alcohol was achieved, but with a low enantioselectivity of 45%. The experimental details are the same as that of platinum nanowire.

### Typical Procedure for Asymmetric Hydrogenation in Water

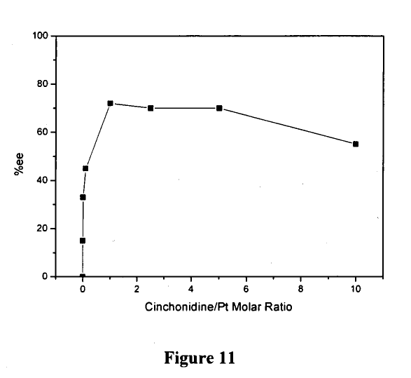

Alkaloid (0.2 mmol) and acetic acid (0.2 mmol) were placed in a 25-mL stainless steel Paar reactor autoclave system, and a slurry of nanowires and nanorods (0.22 mmol) in water was added, followed by ethyl pyruvate (5 mmol) suspended in water (5 mL). The autoclave was closed, and purged with 100 psi (about 690 kPa) of nitrogen three times and then with 100 psi of hydrogen five times. The autoclave was pressurized to 100 psi (about 690 kPa), and the reaction was stirred at room temperature. The reaction was monitored from the pressure decrease in the reactor, and was stopped when the pressure reading became constant. After the completion of reaction, the pressure was released, and the autoclave was purged with nitrogen. The catalyst was removed by centrifugation, and the reaction mixture was analyzed by chiral high-performance liquid chromatography (HPLC). FIG. 10 shows the effect of pressure on this reaction, and FIG. 11 shows the effect of alkaloid concentration on the enantioselectivity of the reaction.

### Asymmetric Hydrogenation of Ketoesters Over Pt-Wire

A variety of alkaloid chiral modifiers were investigated in the asymmetric hydrogenation of ethyl pyruvate, as shown below.

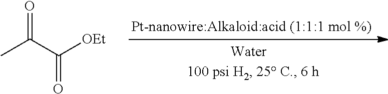

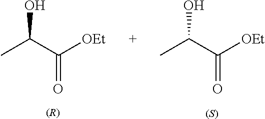

| | 8-(R), 9-(S) | R | Z | 8-(S), 9-(R) | |
|---|---|---|---|---|---|
| 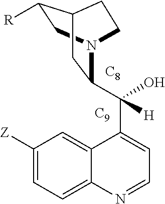 | Cinchonidine (Cd) Dihydrocinchonidine (HCd) Quinine (Qn) Dihydroquinine (HCn) | Vin Et Vin Et | H H OMe OMe | (Cn) Cinchonine (HCn) Dihydrocinchonine (Qd) Quinidine (HQd) Dihydroquinidine | 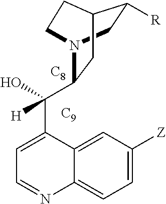 |

The following were observed:
Cd, HCd, Qd, HQd provided (R)-alcohols (quantitative conversions and up to 72% ee)
Cn, HCn, Qn, HQn provided (S)-alcohols (quantitative conversions and up to 70% ee)
Reaction in toluene or ethanol resulted in improved yield and ee relative to other organic solvents. Water-acetic acid was even better than toluene or ethanol.
Slight improvement to % ee (enantiomeric excess) was observed under low pressure at 40 psi (about 275 kPa) relative to the standard conditions of about 100 psi (about 690 kPa), however the reaction rate was somewhat slower.
Variations in reactivity and selectivity were observed with alkaloid to Pt ratio (1:1 was the best ratio tested)
No noticeable effect of moisture and air
Catalyst was recycled 10 times in the hydrogenation of ethyl pyruvate without significant loss in activity and selectivity

### Effect of Purified Alkaloid in Asymmetric Hydrogenation

In the asymmetric hydrogenation of ethyl pyruvate, it was observed that purification and crystallization of Cinchona alkaloid chiral modifiers (to obtain a purity of about 99.5%) improved the enantioselectivity of the hydrogenation by 5-6% (as provided by Aldrich, Cn=85% and Cd=95% purity). Entantiomeric excess of 35-78% ee was achieved when Cd was used, and 30-76% ee when Cn was used (as determined by chiral HPLC).

Asymmetric hydrogenation of dihydro-4,4-dimethyl-2,3-furandione was also performed to produce pantolactone with quantitative conversion and 55% enantioselectivity. This could then be elaborated to produce optically active vitamin B5 (pantothenic acid). Crystallization of the product provided further enantio-enrichment.

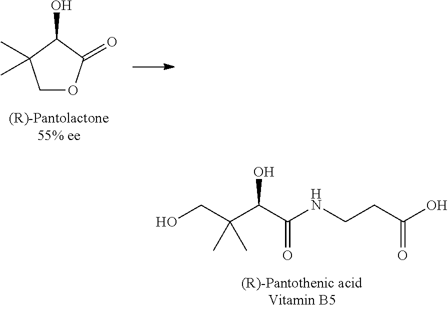

### Asymmetric Hydrogenation of Tosyl Imines

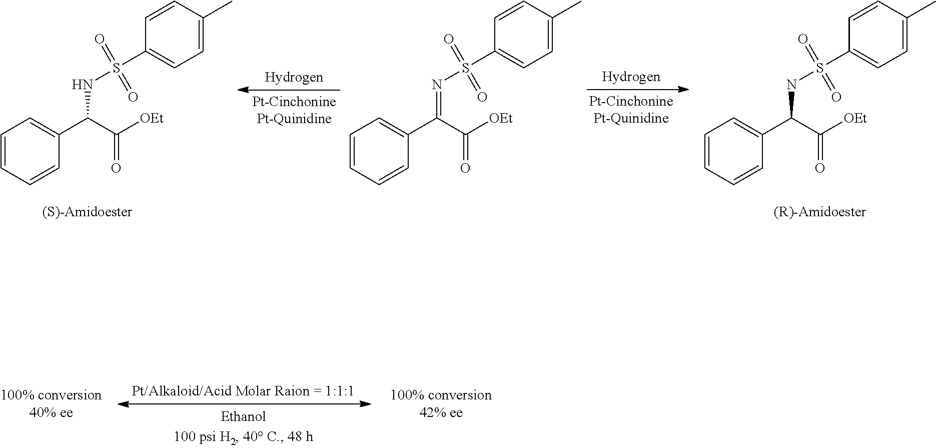

The inventors report asymmetric hydrogenation of tosylimines by Pt nanowires using ammonium formate as a hydrogen source. By contrast, under transfer hydrogenation conditions using KOH/isopropanol at 60° C., quantitative conversion was achieved but with ee less than 15%.

### Pt Nanowire for Transfer Hydrogenation of Ketones

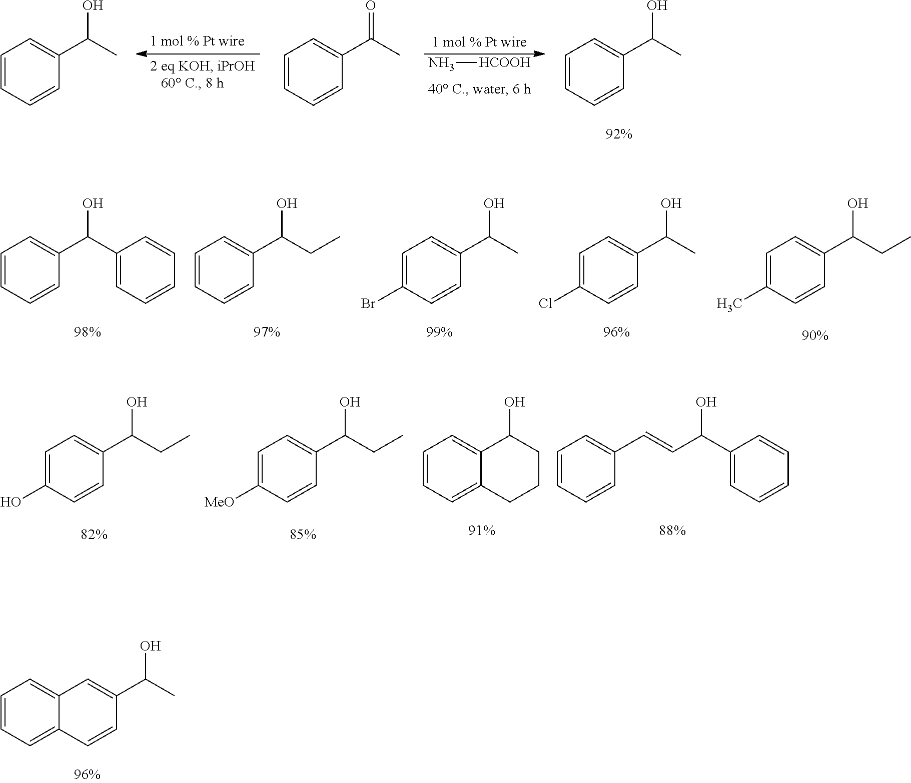

99% conversions by GCMS and TLC were achieved under KOH/isopropanol conditions. Isolated yields are shown above for transfer hydrogenation using ammonium formate in water at 40° C., for 24 h. The catalyst was recovered by centrifugation and reused 3 times for the transfer hydrogenation of propiophenone with similar catalyst activity in each reuse.

### FePt or PtRu Catalyzed Carbenoid Insertions

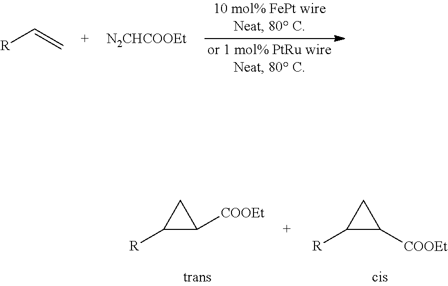

FePt-nanowire-catalyzed intermolecular cyclopropanation
of alkenes with ethyl diazoacetate.

| Entry | Catalyst | R | Yield | trans/cis/% |
|---|---|---|---|---|
| 1 | FePt-nanowire | C6H5 | 78 (98) | 65/35 (75/25) |
| 2 | recycle I | C6H5 | >95 (98) | 66/34 (75/25) |
| 3 | recycle II | C6H5 | >95 (98) | 65/35 (75/25) |
| 4 | recycle III | C6H5 | >95 (98) | 65/35 (75/25) |
| 5 | FePt-nanowire | C6H5 | 65 | 75/25 |
| 6 | FePt particles | C6H5 | 63 | 70/30 |
| 7 | Pt-nanowire | C6H5 | 63 | 70/30 |
| 8 | FePt-nanowire | p-Cl—C6H5 | 40 | 60/40 |
| 9 | FePt-nanowire | p-OCF3—C6H5 | 55 | 55/45 |
| 10 | FePt-nanowire | p-OMe—C6H5 | 71 | 55/45 |
| 11 | FePt-nanowire | n-butyl | 15 | 70/30 |
| 12 | FePt-nanowire | 2-Vinylpyridine | 35 | 50/50 |
| 13 | FePt-nanowire | 4-Vinylpyridine | 32 | 60/40 |

Yield and trans/cis ratios using 1 mol % PtRu catalyst (based on Ru) are shown in the above table for experiments conducted under neat conditions for 1 h at 80° C. The reaction was scaled up to 25 mmol scale using PtRu catalyst in the case of cyclopropanation of styrene.

### Summary

Asymmetric hydrogenation of tosylimines by alkaloid modified Pt-catalyst was demonstrated for the first time. Pt nanowires have been found to be excellent recyclable catalyst for transfer hydrogenation of ketones, e.g. using isopropanol and KOH or ammonium formate in water, and for carbenoid insertion reactions.

1. A nanoparticulate material comprising long ultrathin metal nanowires.

2. The nanoparticulate material of claim 1 wherein the metal nanowires are single crystal metal nanowires.

3. The nanoparticulate material of claim 1 or claim 2 wherein the nanowires have a diameter of less than about 2 nm and a length of greater than about 40 nm.

4. The nanoparticulate material of claim 3 wherein the nanowires have a length of about 100 to about 500 nm.

5. The nanoparticulate material of any one of claims 1 to 4 wherein the nanowires have a diameter of less than or equal to about 1 micron.

6. The nanoparticulate material of any one of claims 1 to 4 wherein the nanowires are straight.

7. The nanoparticulate material of any one of claims 1 to 6 wherein the metal is selected from the Group 8 to Group 11 elements, or is a mixture of any two or more Group 8 to Group 11 elements.

8. The nanoparticulate material of claim 7 wherein the metal is selected from the group consisting of platinum, palladium, rhodium, ruthenium and gold.

9. The nanoparticulate material of claim 8 wherein the metal is platinum.

10. The nanoparticulate material of claim 9 wherein the nanowires have predominant exposure of (111) planes on the surface thereof.

11. The nanoparticulate material of any one of claims 1 to 10 which is catalytic.

12. The nanoparticulate material of claim 11 which is catalytic for a hydrogenation reaction.

13. The nanoparticulate material of claim 11 or claim 12 wherein the metal nanowires have a chiral modifier associated therewith.

14. The nanoparticulate material of claim 13 wherein the chiral modifier is selected from the group consisting of an alkaloid, an optically active aminoalcohol, and optically active amino acid, an optically active diamine, an optically active phosphine and an optically active aminophosphine or is a mixture of any two or more of these.

15. The nanoparticulate material of claim 14 wherein the chiral modifier is selected from the group consisting of 8R,9S-cinchonidine, 8R,9S-dihydrocinchonidine, 8R,9S-quinine, 8R,9S-dihydroquinine, 8S,9R-cinchonidine, 8S,9R-dihydrocinchonidine, 8S,9R-quinine and 8S,9R-dihydroquinine.

16. A process for making a nanoparticulate material comprising:
a) preparing a mixture of a precursor and an amine, said precursor being capable of being converted to a metal or a mixture of metals; and
b) exposing the mixture to a metal carbonyl at elevated temperature;
so as to produce the nanoparticulate material in the form of metal nanowires.

17. The process of claim 16 wherein the precursor is a precursor to a metal selected from the Group 8 to Group 11 elements, or is a mixture of two or more such precursors.

18. The process of claim 17 wherein the precursor, or at least one of the precursors, is a metal complex.

19. The process of claim 18 wherein the complex is an acetylacetone (acac) complex.

20. The process of claim 19 wherein the complex is Pt(acac)₂.

21. The process of any one of claims 16 to 20 wherein the amine is a C6 to C18 amine.

22. The process of any one of claims 16 to 21 wherein the amine is an alkenylamine.

23. The process of claim 22 wherein the amine is oleylamine.

24. The process of any one of claims 16 to 23 wherein step b) is conducted under an inert atmosphere.

25. The process of any one of claims 16 to 24 wherein the metal carbonyl is iron pentacarbonyl.

26. The process of any one of claims 16 to 25 wherein the elevated temperature is between about 100 and about 300° C.

27. The process of any one of claims 16 to 26 additionally comprising the step of treating the nanowires with an etchant capable of removing the metal of the metal carbonyl.

28. The process of claim 27 wherein the etchant is an acid.

29. The process of claim 28 wherein the acid is hydrochloric acid.

30. The process of any one of claims 16 to 29 wherein the mixture produced in step a) also comprises a carboxylic acid salt.

31. The process of claim 30 wherein the carboxylic acid is a C6 to C18 carboxylic acid salt.

32. The process of claim 30 or 31 wherein the carboxylic acid salt is an alkenoic acid salt.

33. The process of claim 32 wherein the carboxylic acid salt is an oleate.

34. The process of any one of claims 16 to 33 additionally comprising exposing the metal nanowires to a chiral modifier.

35. The process of claim 34 wherein the chiral modifier is selected from the group consisting of an alkaloid, an optically active aminoalcohol, an optically active amino acid, an optically active diamine, an optically active phosphine and an optically active aminophosphine or is a mixture of any two or more of these.

36. The process of claim 35 wherein the chiral modifier is selected from the group consisting of 8R,9S-cinchonidine, 8R,9S-dihydrocinchonidine, 8R,9S-quinine, 8R,9S-dihydroquinine, 8S,9R-cinchonidine, 8S,9R-dihydrocinchonidine, 8S,9R-quinine and 8S,9R-dihydroquinine.

37. A method for conducting a catalytic reduction comprising exposing a substrate to a nanoparticulate material according to any one of claims 11 to 15 in the presence of a hydrogen source.

38. The method of claim 37 wherein said nanoparticulate material is made by the process of any one of claims 16 to 36.

39. The method of claim 37 or claim 38 which is conducted in an aqueous solvent.

40. The method of any one of claims 37 to 39 wherein the metal nanowires are selected from the group consisting of platinum nanowires, platinum/ruthenium nanowires and platinum/iron nanowires.

41. The method of any one of claims 37 to 40 wherein the hydrogen source is hydrogen gas.

42. The method of claim 41 wherein the hydrogen gas is at a pressure of less than about 750 kPa.

43. The method of any one of claims 37 to 40 wherein the hydrogen source is ammonium formate.

44. The method of any one of claims 37 to 40 wherein the hydrogen source is alkaline isopropanol.

45. The method of any one of claims 37 to 44 wherein the nanowires of the nanoparticulate substance have a chiral modifier associated therewith, whereby the method produces an optically active product.

46. The method of claim 45 wherein the chiral modifier is a naturally occurring product or a protonated form thereof.

47. The method of claim 45 or 46 wherein the chiral modifier is an alkaloid or a protonated alkaloid.

48. The method of any one of claims 45 to 47 wherein the optically active product has an enantiomeric excess of at least about 50%.

49. The method of any one of claims 37 to 48 which produces a product in at least about 90% chemical yield.

50. The method of any one of claims 37 to 49 comprising reusing the nanoparticulate substance in a subsequent catalytic reduction.

51. Use of a nanoparticulate substance according to any one of claims 11 to 15 in catalysis.

52. The use of claim 51 wherein the catalysis is catalysis of a hydrogenation reaction.
